(19) United States
(12) Patent Application Publication
(10) Pub. No.: US 2007/0207206 A1
(43) Pub. Date: Sep. 6, 2007

(54) Chewable pain formula

(76) Inventor: Bronwyn C. Rice, Portland, OR (US)

Correspondence Address: Ingrid McTaggart, 1816 S.E. 54th Ave., Portland, OR, 97215, US

(21) Appl. No.: 11/367,500

(22) Filed: Mar. 2, 2006

(51) Int. Cl. A61K 9/20; A61K 31/192; A61K 31/16

(52) U.S. Cl. 424/464; 514/569; 514/629

(57) ABSTRACT

One embodiment of a chewable pain relief composition includes acetaminophen, present in a therapeutically-effective amount, ibuprofen, present in a therapeutically-effective amount, and a chewable binding agent, present in an amount effective to facilitate breakdown of the composition during chewing thereof.

### BACKGROUND

Pain medicine may be taken to manage a number of pain symptoms such as post-operative pain, headaches, dental work, swelling and/or fever, and dysmenorrhea. The medications of the prior art do not provide the symptom relief and ease of administration of the present chewable pain formula.

### BRIEF DESCRIPTION OF THE DRAWINGS

FIG. 1 is a schematic perspective view of one embodiment of a chewable pain formula.

FIG. 2 is a flowchart of one embodiment of a process of manufacturing one embodiment of a chewable pain formula.

### DETAILED DESCRIPTION OF THE DRAWINGS

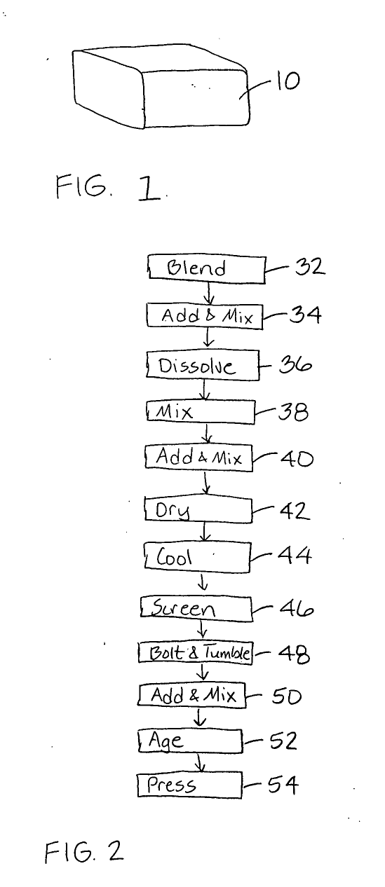

FIG. 1 shows one embodiment of a pain formula in chewable tablet form 10. Chewable tablet 10 may be chewed instead of swallowed as are some prior art pain relief tablets. Many people experience anxiety when swallowing a tablet. Accordingly, chewable tablet 10 of the present invention may be easier to administer than prior art pain relief medications and may reduce the anxiety of certain people when taking the pain formula.

FIG. 2 is a flowchart of one embodiment of a process 12 of manufacturing a chewable pain formula. Process 12 of making chewable tablet 10 may include mixing together one or more pain relief active ingredients 14 and one or more excipients 16, i.e., inactive ingredients. The process shown is only one embodiment. Of course, other embodiments of process 12 may be utilized, such as a different sequence of the mixing steps discussed, and other combinations and amounts of ingredients, which would not change the efficacy of the formula.

Pain relief active ingredient 14 may include ibuprofen, acetaminophen, pyrilamine maleate, pamabrom, aspirin, cimetidine, caffeine, ertyromycin, and magnesium salicylate, or the like, and mixtures thereof. Excipient 16 may include a binder 18, a lubricant 20, a disintegrant 22, a coloring 24, a flavoring 26, a diluent 28, a gum base 30 and mixtures thereof.

Binder 18 may include any material that promotes cohesion of the pain relief formula, such as carbomers, carboxymethylcellulose sodium, ceratonia, confectioner's sugar, cottonseed oil, dextrates, dextrin, dextrose, ethylcellulose, liquid glucose, glyceryl behenate, guar gum, hydrogenated vegetable oil type 1, hydroxyethyl cellulose, hydroxyethylmethyl cellulose, hydroxypropyl cellulose, hypromellose, magnesium aluminum silicate, maltodextrin, maltose, methylcellulose, microcrystalline cellulose, poloxamer, polydextrose, polyethylene oxide, povidone, sodium alginate, pregelatinized starch, starch paste, cellulose, glycol, amylose, polyvinylpyrrolidone, starch, pregelatinized starch, or the like, and mixtures thereof. The cellulose may be a microcrystalline cellulose (MCC) or a colloidal MCC. The glycol may be a sodium starch glycolate or polyethylene glycol.

Lubricant 20 may include any material that speeds production or formation of the tablets such as fatty acids or the like. Lubricant 20 may include calcium stearate, glycerin monostearate, hydrogenated vegetable oil type 1, polyethylene glycol, sodium chloride, sodium stearyl fumarate, talc, zinc stearate, magnesium stearate, and the like, or mixtures thereof.

Disintegrant 22, also referred to as a chewable binding agent or a chewable binder (as opposed to merely a “binder”), may include any material that promotes rapid dispersal or disintegration in the mouth of the pain relief formula once administered, i.e., breakdown of the tablet when chewed so as to provide a chewable tablet. Disintegrant 22 may include cellulose, clays, algins, gums, cross-linked polymers, a cellulose derivative, starches, starch derivatives, for example, corn starch, methylcellulose, sodium lauryl sulfate, sodium starch glycolate, and croscarmellose sodium, carboxymethylcellulose calcium, carboxymethylcellulose sodium, chitosan, colloidal silicon dioxide, croscarmellose sodium, crospovidone, low-substituted hydroxypropyl cellulose, methylcellulose, microcrystalline cellulose, povidone, sodium alginate, pregelatinized starch, mannitol, lactose, sorbitol, sucrose, inositol, or the like, or mixtures thereof. The amount of the disintegrant 22 may range from about 0.1 to about five weight percent of the tablet composition, and may be approximately 2.5 weight percent of the tablet composition. For purposes of this description, weight percent is defined as a weight-in-weight percent, which expresses the number of grams of a constituent in 100 grams of solution.

Coloring 24, if utilized, may include any colors, such as red, yellow, green, orange, blue, brown, or the like, or any FDA approved coloring agent, and mixtures thereof, such that tablet 10 may have any final color as is desired. Examples of colorants include chlorophyll and any colors federally approved for use in food and medicines.

Flavoring 26 may include sugar in a variety of forms, mint, sweetening agents such as aspartame, or the like, and mixtures thereof. Mint may also provide a mild anesthetizing effect on the tongue which may increase the chewability properties of tablet 10. Other flavorings may include confectioner's sugar, ethyl maltol, ethyl vanillin, fumaric acid, malic acid, maltol, menthol, vanillin, wintergreen oil, and the like. The sweetening agent may include acesulfame potassium, alitame, compressible sugar, confectioner's sugar, fructose, liquid glucose, maltitol, saccharin, saccharin sodium, sodium cyclamate, sucralose, sucrose, xylitol, and the like, and mixtures thereof.

Diluent 28 may include a filler which may increase the bulk of tablet 10, such as starch, salt, or the like, or any combination thereof. Diluent 28 may be a compressible sugar, confectioner's sugar, dextrates, dextrose, fructose, lactitol, maltose, sorbitol, sucrose, mannitol, xylitol, and the like, and mixtures thereof.

One or more active ingredients 14 may also be provided in a gum base 30 so as to provide a chewable gum tablet 10 that may be chewed for an extended period of time.

In one embodiment, process 12 may include mixing the following ingredients:

200 milligrams (mg) acetaminophen

100 mg ibuprofen

530 mg disintegrant, such as mannitol

20 mg sodium citrate

500 mg Splenda

500 mg corn starch in 9.8 milliliters (ml) of water

10 mg magnesium stearate

10 mg corn starch

a quantity sufficient (qs) of peppermint oil, for example, approximately one drop

In this example embodiment, the powdered acetaminophen and ibuprofen are mixed until well blended 32, such as mixing for approximately one minute. The mannitol is then added, a little at a time, mixing after each addition, to the mix and blended 34 for approximately one to one and a half minutes. The sodium citrate and the Splenda are then dissolved 36 in one ml of water to give a weight solution of approximately 1,492 mg. One ml of the corn starch and water solution is mixed 38 with the sodium citrate/Splenda solution to give a weight of solution of 2,194 mg and a volume of approximately 5 ml. This solution is added in step 40 to the powdered blend of acetaminophen and ibuprofen and mixed for approximately one minute. The resulting granulation is dried in step 42 at approximately 140 degrees Fahrenheit (° F.) for approximately twenty minutes, or until thoroughly dried. The drying step may take longer for larger quantities of granulation or for different drying temperatures. The blend is then cooled 44 to ambient temperature and screened 46 through a size 20 mesh screen. In step 48 10 mg of corn starch are added and 10 mg of magnesium stearate are then bolted over the dried granulation blend and then the blend is tumbled for approximately one minute. A quantity sufficient (qs) for flavor enhancement and/or an anesthetizing effect of the tongue, such as approximately one drop of peppermint oil, is added in step 50 to the granulation and mixed together. The granulation is aged in step 52 for at least twenty-four hours at an ambient temperature. In one example, a granulation was aged for twenty-five and a half hours at a maximum temperature of 69.4° F. at 51 percent humidity and a minimum temperature of 67.3° F. at forty percent humidity. The granulation is then pressed in step 54 at a pressure of approximately 2,000 pounds with a dwell time of approximately one second or less. This results in a single chewable tablet 10 (FIG. 1) that is palatable to the taste and has a chewable texture and consistency.

The process described above may be scaled upwardly for any amount of tablets. Accordingly, for making 100 tablets, the amount of ingredients listed above is merely multiplied by 100. For making 10,000 tablets, the amount of ingredients listed above is merely multiplied by 10,000. The times required for conducting each step may be changed to produce the desired product.

In another embodiment, process 12 may include mixing the following ingredients:

at least 150 milligrams (mg) acetaminophen

at least 75 mg ibuprofen

at least 500 mg disintegrant, such as mannitol

at least 15 mg sodium citrate

at least 450 mg Splenda

at least 450 mg corn starch in at least 9.0 milliliters (ml) of water

at least 8 mg magnesium stearate

at least 8 mg corn starch

at least 1 drop peppermint oil

In this example embodiment, the powdered acetaminophen and ibuprofen are mixed until well blended, such as mixing for at least one minute. The mannitol is then added to the mix a little at a time, blending after each addition, and blended for at least one minute. The sodium citrate and the Splenda are then dissolved in approximately 0.9 ml of water to give a weight solution of approximately 1,369 mg. Slightly less than one ml of the corn starch and water solution is mixed with the sodium citrate/Splenda solution to give a weight of solution of 2,175 mg and a volume of approximately 3.2 ml. This solution is added to the powdered blend of acetaminophen and ibuprofen and mixed for at least one minute. The resulting granulation is dried thoroughly at a temperature of at least 125 degrees Fahrenheit (° F.) for at least one minute. The blend is then cooled to ambient temperature and screened through a size 20 mesh screen. At least 8 mg of corn starch is added and at least 8 mg of magnesium stearate is then bolted over the dried granulation blend and tumbled for at least one minute. At least one drop of peppermint oil is added to the granulation and mixed together. The granulation is aged for at least twenty-four hours at an ambient temperature at a humidity of at most 60 percent and no lower than thirty percent. The granulation is then pressed at a pressure of at least 1,500 pounds with a dwell time of approximately one second or less. This results in a single chewable tablet 10 that is palatable to the taste and has a chewable texture and consistency.

There may be other embodiments in which all ingredients may be simultaneously blended together to form a wet blend. The blend may then be dried, cooled, granulated and pressed to form tablet 10 without the discriminate blending steps described above.

The process of the present invention, in one embodiment, produces a chewable tablet 10 (FIG. 1) including both acetaminophen and ibuprofen. Inclusion of these two different types of pain relief medication is thought by Applicant to provide advantages over pain relief medications including only one or the other of such pain relief ingredients. In particular, acetaminophen appears to work at the nerve endings in areas where an individual is experiencing pain by blocking the nerve impulses that signal the pain to the brain. It is believed to produce analgesia by elevating the pain threshold. Ibuprofen appears to inhibit prostaglandin synthesis and, therefore, is effective as a pain reliever in prostaglandin-mediated processes, such as dysmenorrhea. Accordingly, combining acetaminophen and ibuprofen is thought to provide increased efficacy in pain management.

Moreover, ibuprofen is an inhibitor of the COX1 and COX2 enzymes. The COX1 enzyme protects the stomach lining and other mucosal material in the body. Accordingly, ibuprofen may damage the stomach and kidneys when used in excessive amounts. Acetaminophen may damage the liver when used in excessive amounts. Applicant's combination of acetaminophen and ibuprofen in a single tablet, therefore, may allow reduced amounts of each pain relief ingredient in each dose so as to reduce potential harm to the body's respective organs, namely, the stomach, kidneys and liver. Combining the two pain relief ingredients in a single tablet may also reduce the amount of each ingredient utilized in each tablet thereby reducing the risk of overdosing that may occur when the two drugs are used separately in an alternating manner.

Other variations and modifications of the concepts described herein may be utilized and fall within the scope of the claims below.

We claim:

1. A chewable pain relief composition, comprising:
acetaminophen, present in a therapeutically-effective amount;
ibuprofen, present in a therapeutically-effective amount; and
a chewable binding agent, present in an amount effective to facilitate breakdown of said composition during chewing thereof.

2. The composition of claim 1 wherein said chewable binding agent is chosen from one of cellulose, a clay, algin, gum, a cross-linked polymer, a cellulose derivative, a starch, a starch derivative, corn starch, methylcellulose, sodium lauryl sulfate, sodium starch glycolate, croscarmellose sodium, carboxymethylcellulose calcium, carboxymethylcellulose sodium, chitosan, colloidal silicon dioxide, croscarmellose sodium, crospovidone, low-substituted hydroxypropyl cellulose, methylcellulose, microcrystalline cellulose, povidone, sodium alginate, pregelatinized starch, mannitol, lactose, sorbitol, sucrose and inositol.

3. The composition of claim 1 further comprising a lubricant chosen from one of calcium stearate, glycerin monostearate, hydrogenated vegetable oil type 1, polyethylene glycol, sodium chloride, sodium stearyl fumarate, talc, zinc stearate and magnesium stearate.

4. The composition of claim 1 further comprising a coloring chosen from one of any FDA approved coloring agent.

5. The composition of claim 1 further comprising a flavoring chosen from one of sugar, mint, aspartame, confectioner's sugar, ethyl maltol, ethyl vanillin, fumaric acid, malic acid, maltol, menthol, vanillin, wintergreen oil, acesulfame potassium, alitame, compressible sugar, confectioner's sugar, fructose, liquid glucose, maltitol, saccharin, saccharin sodium, sodium cyclamate, sucralose, sucrose and xylitol.

6. The composition of claim 1 further comprising a diluent chosen from one of compressible sugar, confectioner's sugar, dextrate, dextrose, fructose, lactitol, maltose, sorbitol, sucrose, mannitol and xylitol.

7. The composition of claim 1 wherein said composition comprises a tablet and wherein said chewable binding agent is present in an amount of at least 2.5 weight percent of said tablet.

8. The composition of claim 1 wherein said composition comprises a tablet and wherein said ibuprofen is present in a therapeutically-effective amount of at least 75 mg per tablet.

9. The composition of claim 1 wherein said composition comprises a tablet and wherein said acetaminophen is present in a therapeutically-effective amount of at least 150 mg per tablet.

10. A chewable pain relief composition, comprising:
acetaminophen;
ibuprofen; and
a chewable binder that breaks down upon chewing so as to orally release said acetaminophen and ibuprofen.

11. The composition of claim 10 wherein said chewable binder is chosen from the group including cellulose, a clay, algin, gum, a cross-linked polymer, a cellulose derivative, a starch, a starch derivative, corn starch, methylcellulose, sodium lauryl sulfate, sodium starch glycolate, croscarmellose sodium, carboxymethylcellulose calcium, carboxymethylcellulose sodium, chitosan, colloidal silicon dioxide, croscarmellose sodium, crospovidone, low-substituted hydroxypropyl cellulose, methylcellulose, microcrystalline cellulose, povidone, sodium alginate, pregelatinized starch, mannitol, lactose, sorbitol, sucrose and inositol.

12. The composition of claim 10 wherein said chewable binder comprises at least 2.5 weight percent of said composition.

13. The composition of claim 10 wherein said acetaminophen comprises at least 150 mg per single dose of said composition.

14. The composition of claim 10 wherein said ibuprofen comprises at least 75 mg per single dose of said composition.

15. A method of manufacturing a chewable pain relief tablet, comprising:
blending acetaminophen, ibuprofen and a disintegrant together to provide a blend; and
pressing said blend to produce a chewable tablet.

16. The method of claim 15 wherein said pressing is conducted at a pressure of at least 1,500 pounds.

17. The method of claim 15 wherein said disintegrant is chosen from at least one of cellulose, a clay, algin, gum, a cross-linked polymer, a cellulose derivative, a starch, a starch derivative, corn starch, methylcellulose, sodium lauryl sulfate, sodium starch glycolate, croscarmellose sodium, carboxymethylcellulose calcium, carboxymethylcellulose sodium, chitosan, colloidal silicon dioxide, croscarmellose sodium, crospovidone, low-substituted hydroxypropyl cellulose, methylcellulose, microcrystalline cellulose, povidone, sodium alginate, pregelatinized starch, mannitol, lactose, sorbitol, sucrose and inositol.

18. The method of claim 15 further comprising, prior to said pressing, blending said acetaminophen and ibuprofen in powder form, adding said disintegrant and a binder to provide said blend, drying said blend, and then screening said blend to form a granulation.
